(19) United States
(12) Patent Application Publication
(10) Pub. No.: US 2006/0241708 A1
(43) Pub. Date: Oct. 26, 2006

(54) Multiple sensors for sleep apnea with probability indication for sleep diagnosis and means for automatic activation of alert or therapy

(76) Inventor: Willem Boute, Dieren (NL)

Correspondence Address: MEDTRONIC, INC., 710 MEDTRONIC PARK, MINNEAPOLIS, MN, 55432-9924, US

(21) Appl. No.: 11/112,425

(22) Filed: Apr. 22, 2005

(51) Int. Cl. A61N 1/00; A61B 5/08

(52) U.S. Cl. 607/17; 607/18; 607/22; 600/529

(57) ABSTRACT

An apparatus and method for detecting respiratory disturbances based on multiple physiological parameters are provided. The method includes sensing one or more physiological signals, deriving from the sensed signals multiple physiological parameters that change during a respiratory disturbance, computing a probability that the respiratory disturbance is present using the multiple physiological parameters, and detecting the respiratory disturbance if the probability exceeds a predetermined threshold. In some embodiments, the method further includes generating an alert signal or other report in response to a respiratory disturbance detection. In other embodiments, the method further includes triggering the delivery of a therapy in response to a respiratory disturbance detection.

### FIELD OF THE INVENTION

The present invention relates generally to medical devices and in particular to a device and method for detecting and treating sleep apnea.

### BACKGROUND OF THE INVENTION

Central or obstructive forms of sleep apnea syndrome are prevalent in both normal and heart failure populations. Respiratory disturbances are associated with a number of pathological conditions. Cheyne-Stokes respiration is the waxing and waning of respiration associated with congestive heart failure. Kussmaul breathing is rapid deep breathing associated with diabetic ketoacidosis. Detection of respiratory disturbances, such as sleep apnea, Cheyne-Stokes respiration, Kussmaul breathing, or other disordered breathing, may be useful in monitoring a patient's disease status, selecting treatment and monitoring its effectiveness.

A standard diagnostic approach for sleep apnea includes polysomnography, which requires the patient to stay overnight in a hospital for observation, in addition to medical history and screening questionnaires. Polysomnography involves monitoring of multiple parameters including electroencephalography, electromyography, electrocardiography, oximetry, airflow, respiratory effort, snoring, body position and blood pressure. Polysomnography measures a patient's respiratory patterns during a single sleeping period and is expensive and inconvenient for the patient. A single evaluation of the patient's sleep patterns may not be adequate to detect and diagnose a problem. Furthermore, a physician must actively prescribe the sleep study and therefore must already suspect a sleep-related breathing disorder.

Respiratory disturbances in the form of sleep-related disordered breathing often go undetected in patients suffering from heart failure or sleep apnea. Nocturnal Cheyne-Stokes respiration, a form of central sleep apnea, occurs frequently in patients with chronic heart failure. The presence of sleep apnea significantly worsens the prognosis for a heart failure patient. Therefore, recognizing and monitoring the presence of disordered breathing in heart failure patients could provide useful diagnostic and prognostic information and may initiate and steer therapies for breathing disorders.

Monitoring of respiratory disturbances is also desirable in diabetic patients. Diabetic ketoacidosis may be the first symptom to appear in a person with Type I diabetes. Diabetic ketoacidosis develops when blood is more acidic than body tissues due to the accumulation of ketones in the blood when body fat is metabolized for energy in place of glucose reserves when insulin is not available. Persons having Type II diabetes usually develop ketoacidosis only under conditions of severe stress. Recurrent episodes of ketoacidosis in diabetic persons are generally the result of poor compliance with dietary restrictions or self-administered treatments. Kussmaul breathing is a common symptom of ketoacidosis. Therefore early detection and monitoring of Kussmaul breathing in diabetic patients may be valuable in the effective control of diabetes. Respiratory monitoring may be a preferred method for monitoring diabetic status in combination with or in place of periodically measuring blood glucose, which requires the use of hypodermic needles with associated risks of infection or contamination.

Respiration may be measured directly using, for example, external breathing masks equipped with airflow sensors or other types of sensors for sensing respiration. Breathing masks, however, are generally not well tolerated by patients for extended periods of time. It is desirable to provide a system and method that is easily tolerated by the patient for detecting and monitoring episodes of respiratory disturbances, which disturbances may be associated with a particular pathological condition. Monitoring of respiratory disturbances may be valuable in the diagnosis, prognosis, and therapy management of a patient.

### BRIEF SUMMARY OF THE INVENTION

The invention provides an apparatus and method for detecting episodes of a respiratory disturbance based on multiple physiological parameters. The method includes sensing one or more physiological signals, deriving from the sensed signals multiple physiological parameters that change during a respiratory disturbance, determining the probability that the respiratory disturbance is present using the multiple physiological parameters, and detecting the respiratory disturbance if the probability exceeds a predetermined threshold. In some embodiments, the method further includes generating an alert signal or other report in response to a respiratory disturbance detection. In other embodiments, the method further includes triggering the delivery of a therapy in response to a respiratory disturbance detection.

The apparatus for respiratory disturbance detection may be an implantable or external medical device system. The apparatus includes one or more physiological sensors coupled to signal processing circuitry for deriving multiple physiological parameters. The apparatus further includes processing circuitry for receiving the physiological parameters, computing a respiratory disturbance probability using the physiological parameters, and generating a respiratory disturbance detection signal if the probability exceeds a predetermined threshold. The apparatus may further include alert circuitry for generating a patient or physician alert, which may include the transfer of data via a communication link or network, in response to a respiratory detection signal. In other embodiments, the apparatus may further include therapy control and delivery circuitry for delivering a therapy in response to a respiratory disturbance detection signal.

Another aspect of the invention is a set of instructions stored on a computer-readable medium which, when implemented by a medical device causes the device to derive multiple physiological parameters from one or more physiological signal sources, compute a respiratory disturbance probability form the physiological parameters, compare the respiratory disturbance probability to a detection threshold, and generate a response to a respiratory disturbance detection.

### BRIEF DESCRIPTION OF THE DRAWINGS

FIG. 1 is an illustration of one type of a medical device in which the invention may be implemented.

FIG. 2 is a block diagram summarizing the data acquisition and processing functions included in the medical device shown in FIG. 1.

FIG. 3 is a flow chart summarizing one method for detecting sleep apnea using multiple physiological signals.

FIG. 4 is a flow chart summarizing steps included in a method for responding to a sleep apnea detection made according to the method of FIG. 3.

### DETAILED DESCRIPTION

The invention provides a method and apparatus for detecting a respiratory disturbance and providing a response thereto. The invention may be implemented in implantable medical devices (IMDs) that include sensing capabilities for monitoring a physiological condition and may include therapy delivery capabilities. An IMD in which the invention is implemented may be primarily intended for monitoring respiratory disturbances for diagnostic or prognostic purposes. In one embodiment, an IMD may be primarily intended for monitoring for sleep apnea. The IMD may alternatively be intended primarily for detecting and treating sleep apnea. IMDs used for treating sleep apnea may deliver a sleep apnea therapy in the form of cardiac overdrive pacing or neuromuscular stimulation such as pectoral stimulation, phrenic nerve stimulation, or stimulation of excitable tissue in the neck or throat. An IMD may, via telemetry, trigger an external system to generate a patient alert or deliver a therapy or for transmitting an alert signal to a clinician or medical facility via wireless or wired communications network.

The invention may alternatively be implemented in IMDs that are used primarily for other monitoring and/or therapy delivery purposes. Appropriate IMDs in which the invention may be incorporated include, but are not limited to, cardiac pacemakers, implantable cardioverter defibrillators (ICDs), cardiac monitoring devices, neuromuscular stimulators and drug pumps. The inclusion of respiratory disturbance detection in such devices can improve the therapeutic, diagnostic and/or prognostic usefulness of the device when the respiratory disturbance is associated with the primary condition being monitored or treated by the IMD, such as heart failure or diabetes.

The invention may also be implemented in external medical devices. External medical devices may be used for bedside monitoring of a patient for diagnosing and/or treating sleep apnea or another medical condition that can be associated with respiratory disturbances. For example, external continuous positive airway pressure (CPAP) devices are used for detecting sleep apnea and providing positive pressure to open the airways in patients having obstructive sleep apnea. External devices used for monitoring heart failure patients may incorporate respiratory disturbance detection methods provided by the present invention for use as a prognostic indicator.

In the description that follows, various embodiments of the invention are described relating to the detection of sleep apnea. The methods and apparatus provided by the present invention, however, are not limited to the detection of sleep apnea but may be used for the detection of other types of respiratory disturbances, such as Cheyne-Stokes breathing or Kussmaul breathing.

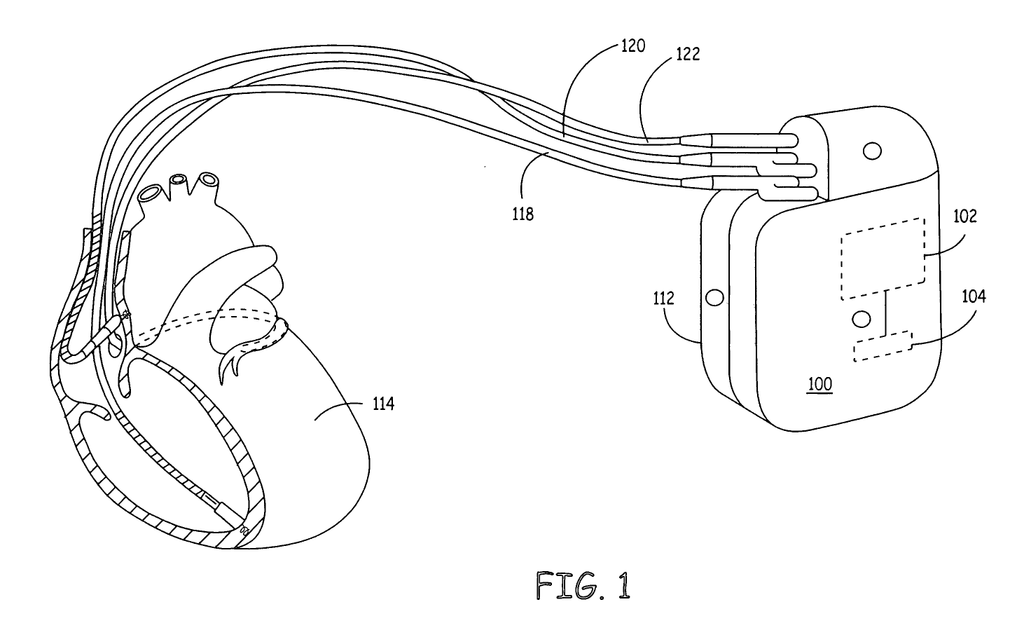

FIG. 1 is an illustration of one type of a medical device in which the invention may be implemented. IMD 100 is shown as an implantable cardiac stimulation device coupled to a set of cardiac leads used for positioning electrodes and other physiological sensors relative to a patient's heart 114 or in the blood volume. IMD 100 may be configured to integrate both monitoring and therapy features, as will be described below. IMD 100 collects and processes data from one or more sensors for deriving parameters used in computing a probability of a respiratory disturbance, such as sleep apnea. IMD 100 may further provide therapy or other response to the patient as appropriate, and as described more fully below.

IMD 100 is provided with a hermetically-sealed housing 112 that encloses a processor 102, a digital memory 104, and other components as appropriate to produce the desired functionalities of the device. In various embodiments, IMD 100 is implemented as any implanted medical device capable of measuring physiological signals for use in detecting sleep apnea or other respiratory disturbances, including, but not limited to a pacemaker, defibrillator, electrocardiogram monitor, blood pressure monitor, drug pump, insulin monitor, or neurostimulator.

Processor 102 may be implemented with any type of microprocessor, digital signal processor, application specific integrated circuit (ASIC), field programmable gate array (FPGA) or other integrated or discrete logic circuitry programmed or otherwise configured to provide functionality as described herein. Processor 102 executes instructions stored in digital memory 104 to provide functionality as described below. Instructions provided to processor 102 may be executed in any manner, using any data structures, architecture, programming language and/or other techniques. Digital memory 104 is any storage medium capable of maintaining digital data and instructions provided to processor 102 such as a static or dynamic random access memory (RAM), or any other electronic, magnetic, optical or other storage medium.

As further shown in FIG. 1, IMD 100 may receive one or more cardiac leads for connection to circuitry enclosed within the housing 112. In one embodiment, IMD 100 collects cardiac electrogram (EGM) signals for use in deriving one or more heart rate related parameters and/or one or more Q-T interval related parameters for use in computing a probability of sleep apnea. In the example of FIG. 1, IMD 100 receives a right ventricular endocardial lead 118, a left ventricular coronary sinus lead 122, and a right atrial endocardial lead 120, although the particular cardiac leads used can vary from embodiment to embodiment. Other lead systems can be substituted for the lead system shown in FIG. 1 and may include auxiliary leads that measure breathing or minute ventilation through impedance changes. In addition, the housing 112 of IMD 100 may function as an electrode and be used for sensing EGM signals. In alternate embodiments, cardiac sensing electrodes may be provided on subcutaneous electrodes located on housing 112 or on subcutaneous leads extending from IMD 100 for sensing ECG signals.

Ventricular leads 118 and 122 may include, for example, pacing electrodes and defibrillation coil electrodes (not shown) in the event IMD 100 is configured to provide pacing, cardioversion and/or defibrillation. In addition, ventricular leads 118 and 122 may deliver pacing stimuli in a coordinated fashion to provide biventricular pacing, cardiac resynchronization, extra systolic stimulation therapy or other benefits. Atrial lead 120 may include pacing electrodes for providing atrial pacing pulses. In one embodiment of the invention, atrial lead 120 is used to provide atrial overdrive pacing in response to sleep apnea detection.

Electrodes carried on leads 118, 120 and 122 or the housing 112 or other auxiliary leads extending from IMD 100 may also be used for measuring impedance signals. Impedance signals are used in deriving respiration-related parameters for use in computing a sleep apnea or other respiratory disturbance probability. The use of impedance signals for monitoring respiration rate and minute ventilation is known in the art, for example in rate responsive cardiac pacemakers.

IMD 100 may obtain other physiological signals used in detecting sleep apnea or other respiratory disturbances. IMD 100 may obtain blood pressure signals, blood oxygen saturation signals, acoustical signals, or other physiological signals for deriving multiple parameters used in computing sleep apnea probability. In one embodiment, IMD 100 receives physiological signals for deriving a heart rate variability, a Q-T interval variability, respiration rate, respiration depth, and blood oxygen saturation. IMD 100 may receive physiological signals from sensors deployed on any of leads 118, 120 and 122 or other auxiliary cardiac or subcutaneous leads or included on or in IMD housing 112.

In operation, IMD 100 obtains data via electrodes and/or sensors deployed on leads 118, 120, 122, and/or other sources. This data is provided to processor 102, which suitably analyzes the data, stores appropriate data in memory 104, and/or provides a response or report as appropriate. Any identified respiratory disturbance episodes can be responded to by intervention of a physician or in an automated manner. In various embodiments, IMD 100 activates an alert upon detection of a respiratory disturbance. Alternatively or in addition to alert activation. IMD 100 selects or adjusts a therapy and coordinates the delivery of the therapy by IMD 100 or another appropriate device, which could be another IMD or an external device adapted to communicate with IMD 100 and respond to a sleep apnea signal from IMD 100. Communication between IMD 100 and another device can occur via telemetry, such as a long-distance telemetry system. Optional therapies that may be applied in response to sleep apnea detection in various embodiments may include overdrive pacing, neuromuscular stimulation, and continuous positive airway pressure.

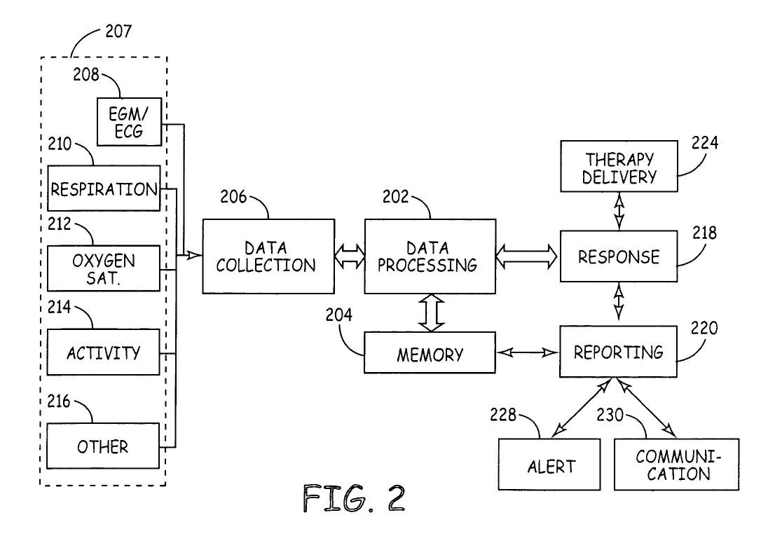

FIG. 2 is a block diagram summarizing the data acquisition and processing functions included in IMD 100. IMD 100 includes a data collection module 206, a data processing module 202, a response module 218 and/or a reporting module 220. Each of the various modules may be implemented with computer-executable instructions stored in memory 104 and executing on processor 102 (shown in FIG. 1), or in any other manner. The exemplary modules and blocks shown in FIG. 2 are intended to illustrate one logical model for implementing an IMD 100 for monitoring respiratory disturbances using multiple physiological signals, and should not be construed as limiting. Indeed, the various practical embodiments may have widely varying software modules, data structures, applications, processes and the like. As such, the various functions of each module may in practice be combined, distributed or otherwise organized in any fashion in or across a medical device system that includes physiological signal sources.

Data collection module 206 is interfaced with one or more data sources 207 to obtain data about the patient. Data sources 207 are generally embodied as sensors that can monitor electrical, mechanical, chemical, or optical information that contains physiological data of the patient. Data sources 207 include any source of physiological signals used for monitoring for a respiratory disturbance or any other physiological event or condition. Data sources 207 include an ECG or EGM source 208 that provides cardiac electrical signals such as P-waves, R-waves or T-waves used to monitor the patient's heart rhythm or conduction times. Data sources 207 further include a respiration signal source 210 for determining respiration rate and depth that can be used for minute ventilation computations. Respiration signal source 210 may be provided as an impedance signal obtained from cardiac electrodes or auxiliary electrodes, for example in the manner used for determining minute ventilation in rate responsive pacemakers. Respiration signal source 210 may alternatively be provided as any physiological signal that varies in response to the respiration cycle.

Data sources 207 further includes a blood oxygen saturation source 212 for monitoring decreases in oxygen saturation that may be indicative of sleep apnea. An activity sensor 214 may be provided which generates a signal responsive to patient activity level and can be used in detecting a rest or sleep state.

Data sources 207 may include other physiological signal sources 216 for acquiring physiological signals useful in monitoring a patient. Other sources 216 may include, for example, an accelerometer or heart wall motion sensor, a blood pressure sensor, a position sensor or a pH sensor. Physiological parameters used for detecting sleep apnea or another respiratory disturbance may be determined from these alternative signal sources. For example, heart rate may be determined from an EGM/ECG signal 208 but may alternatively be determined from a blood pressure signal, a wall motion signal or other heart signal if EGM/ECG source 208 is not available. The various data sources 207 may be provided alone or in combination with each other, and may vary from embodiment to embodiment.

Data collection module 206 receives data from each of the data sources 207 by polling each of the sources 207, by responding to interrupts or other signals generated by the sources 207, by receiving data at regular time intervals, or according to any other temporal scheme. Data may be received at data collection module 206 in digital or analog format according to any protocol. If any of the data sources generate analog data, data collection module 206 translates the analog signals to digital equivalents using an analog-to-digital conversion scheme. Data collection module 206 may also convert data from protocols used by data sources 207 to data formats acceptable to data processing module 202, as appropriate.

Data processing module 202 is any circuit, programming routine, application or other hardware/software module that is capable of processing data received from data collection module 206. In various embodiments, data processing module 202 is a software application executing on processor 102 (FIG. 1) to implement the processes described below for detecting sleep apnea. Accordingly, data processing module 202 processes data received from sources 207 for computing a probability of sleep apnea, as described more fully below, or another respiratory disturbance.

In an exemplary embodiment, processing module 202 receives data from respiration source 210, EGM/ECG source 208, and oxygen saturation source 212 from data collection module 206 and interprets the data using digital signal processing techniques to derive certain information from these sources for computing a probability of sleep apnea. The sleep apnea probability and/or intermediate computational results may be stored in memory 204, which may correspond to hardware memory 104 shown in FIG. 1, or may be implemented with any other available digital storage device. Data storage allows a clinician to access information from the various separate data sources over time and from any combination of these sources over time. This data can be valuable to a clinician, even when sleep apnea is not detected based on the computed sleep apnea probability, since the data can provide insight on the progression of a respiratory disturbance, even when the respiratory disturbance is not yet symptomatic.

When the computed sleep apnea probability exceeds a predetermined threshold, processing module 202 may trigger an appropriate response. Responses may be activated by sending a digital message in the form of a signal, passed parameter or the like to response module 218. Response module 218 is any circuit, software application or other component that interacts with any type of therapy-delivery system 224 and/or reporting module 220. In some embodiments, therapy delivery system 224 is provided as a pulse generating device integrated with IMD 100 to deliver overdrive cardiac pacing or other neuromuscular stimulation in response to sleep apnea detection. Any therapy provided may be controlled or adjusted in response to a sleep apnea detection made using physiological signals acquired by data sources 207.

Reporting module 220 is any circuit or routine capable of producing appropriate feedback from the medical device to the patient or to a clinician or other caregiver. In various embodiments, suitable reports might include storing data in memory 204; generating an alert 228; or producing a communication for transmission from a telemetry circuit or other communication module 230. Communication module 230 may be provided as a hardwired or wireless communication network interface that can be used to transfer an alert or report to a designated recipient via a network, which may be telephone network, local area network, or the like. Reports may include information about sleep apnea episode detections such as the time, date and duration and the severity of the episode, the physiological data collected, and any other appropriate data.

An alert generated by the IMD or an external device responsive to a telemetry signal received from the IMD can be directed to the patient, e.g. as an audible sound, vibration, perceivable muscle stimulation or other sensory alert. An alert may alternatively be directed to a clinician in form of a visual display and/or audible signal. An external device receiving an alert signal from IMD 100 may display recommended actions to be taken by the patient or a caregiver. The external device may include processing circuitry for interpreting data received from the implanted device or transfer data to an expert patient management system containing knowledge that is captured from general therapy protocol of physicians dealing with these respiration disturbances.

An alert signal may result in the telemetry uplink of data obtained from the various sensors to a networked external device (such as a home monitor, personal computer, or cell phone). As such, communication module 230 may include telemetry circuitry for transmitting data from an IMD to an external device adapted for bidirectional telemetric communication with the IMD. The external device receiving the wireless message may be a programmer/monitor device that advises the patient, a physician or other attendant of the sleep apnea detection or related data. Information stored in memory 204 may be provided to an external device to aid in diagnosis or treatment of the patient. Alternatively, the external device may be an interface to a communications network such that the IMD is able to transfer sleep apnea data to an expert patient management center. The external device may transmit data to an expert data management center programmed to process the data and retrieve relevant information for distribution to a clinician, medical center, and/or back to the patient.

The various components and processing modules shown in FIG. 2 may be housed in a common housing such as that shown in FIG. 1. Alternatively, portions of the components and processing modules may be housed separately. For example, portions of the therapy delivery system 224 could be integrated with IMD 100 or provided in a separate housing or as an external device. In this case, response module 218 may interact with therapy delivery system 224 via an electrical cable or wireless link.

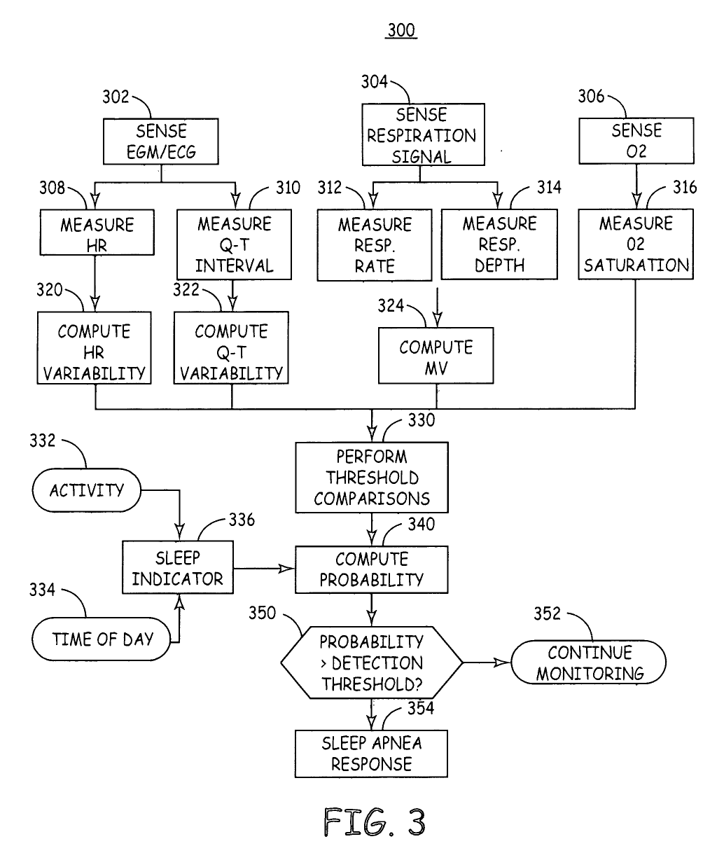

FIG. 3 is a flow chart summarizing one method 300 for detecting sleep apnea using multiple physiological signals. Sleep apnea monitoring according to method 300 may be performed continuously, or on a scheduled or triggered basis. For example, method 300 may be programmed to operate during nighttime hours, when a patient is expected to be asleep, and/or when a position sensor indicates a supine position. Method 300 may additionally or alternatively be enabled to be performed upon a triggering condition. A triggering condition may be a sleep indicator based on an activity signal, posture signal, time of day, or other physiological signal or any combination thereof. Methods for determining or detecting a sleep state are known in the art. Reference is made, for example, to U.S. Pat. No. 6,731,984, issued to Yong, et al. A triggering condition may alternatively be a threshold crossing of any of the physiological signals used in detecting sleep apnea or any combination of those signals, such as a heart rate, a respiration rate or depth, minute ventilation, or blood oxygen saturation level.

Sleep apnea monitoring begins by sensing an EGM/ECG signal at step 302, a respiration signal at step 304, and a blood oxygen saturation signal at step 306. Each of these signals are sensed simultaneously to allow multiple, concurrent physiological parameter values to be determined for use in sleep apnea detection. In some embodiments, the medical device may not be capable of simultaneous sensing and processing of all signals in which case sequential sensing and processing may be performed but may be less sensitive or have a slower response time for sleep apnea detection.

The physiological signals are used for computing a number of parameters that will be used to calculate a sleep apnea probability. At step 308, the EGM/ECG signal is used to measure heart rate. The measured heart rate (HR) is used to compute parameters related to HR such as the HR variability at step 320. HR variability may be computed according to methods known in the art. It is recognized that heart rate and heart rate variability parameters can be determined from alternative cardiac-related signals, such as blood pressure. HR variability or other HR related parameters may become abnormal or otherwise change in a characteristic way at the onset, during, or just after a respiratory disturbance.

At step 310, the EGM/ECG signal is used to measure Q-T intervals. The Q-T interval variability, QT rate dependency, the absolute length of the QT interval or any other QT related parameter can be computed at step 322 using the measured Q-T intervals. The Q-T interval and/or its relation to HR may change in a characteristic manner at the onset, during or just after a sleep apnea episode and therefore be useful in sleep apnea detection or confirmation.

The respiration signal sensed at step 304, which may be an impedance signal, is used to measure the respiration rate at step 312 and the respiration depth at step 314. Respiration rate and depth may be measured on a cycle-by-cycle basis or as mean or median value determined from a predetermined number of successive respiration cycles. The respiration rate and depth are used at step 324 for computing minute ventilation (MV). A low respiration rate and/or low respiration depth, and/or low minute ventilation occurs during sleep apnea.

The oxygen saturation signal sensed at step 306 is used to measure the oxygen saturation level at step 316. The oxygen saturation signal may be averaged over a predetermined interval of time for determining the oxygen saturation level at step 316. A decrease in oxygen saturation can be a result of sleep apnea.

At step 330, method 300 may perform threshold comparisons of one or more of the measured parameters. Threshold values that would be indicative of a sleep apnea episode may be predefined for any of the measured parameters.

At step 340, the parameter values and/or threshold comparison results are used in computing a sleep apnea probability. The measured or computed parameter value may be used in computing the probability at step 340. Alternatively, the result of a threshold comparison for any given parameter value may be used. For example, if the oxygen saturation level goes below a threshold value, the oxygen saturation parameter may be assigned a logical value of 1, indicating the oxygen saturation parameter is positive for sleep apnea detection. If the oxygen saturation level remains or returns to a value above the threshold value, the oxygen saturation parameter may be assigned a logical value of 0, indicating the oxygen saturation parameter is negative for sleep apnea detection. Each of the monitored parameters may be assigned a weighting coefficient used in computing the sleep apnea probability at step 340. A positive indication for sleep apnea may therefore be derived from a change in one or more parameter values and/or from a threshold crossing of one or more parameter values.

A sleep indicator determined at step 336 may also be used in computing the sleep apnea probability at step 340. A sleep indicator may be based on an activity sensor signal 332 and/or the time of day 334. If the activity level is below a threshold level and the time of day is nighttime, the sleep indicator is positive. Other methods known in the art for detecting a sleep state may be used.

In one embodiment, the sleep apnea probability (SAP) computed at step 340 is computed according to the following equation:
SAP=a(HRV)+b(QTV)+c(RR)+d(RD)+f(MV)+g(O2sat)+h(SI)

wherein HRV is the measured heart rate variability or the logical result of a threshold comparison of the HR variability to a predetermined threshold. QTV is the measured Q-T interval variability or the logical result of a threshold comparison of Q-T interval variability to a predetermined threshold. RR is the respiration rate, RD is the respiration depth, and MV is minute ventilation. O2sat is the oxygen saturation level, and SI is the sleep indicator. The values used for each of these parameters may be a measured or computed value or a logical value based on the results of a threshold comparison performed at step 330. The constants a, b, c, d, f, g, and h are weighting coefficients that may be any predefined value including 0. The appropriate values for the weighting coefficients may be determined through optimization techniques applied to individual patients to maximize the sensitivity and specificity of sleep apnea detection.

The weighting coefficient values may alternatively be based on historical clinical experience. For example, the coefficient values may be derived from the long term storage of individual sensor data. The clinician can review the sensor data for a given patient and determine correlations between monitored parameter values and periods of sleep apnea. Automatic learning algorithms may be implemented for automatically adjusting the coefficients, for example, based on the composite result of all the sensor signals. Typically, an automatic learning algorithm will require one or more sleep apnea episodes to be confirmed by the patient or a caregiver. Manual conformation can be entered into the system using an external patient device or programmer and communicated to the IMD through telemetry. The coefficients can then be preset to values that would result in a positive sleep apnea detection during the confirmed sleep apnea episode.

At step 350, method 300 determines if the sleep apnea probability exceeds a predetermined sleep apnea detection threshold. If the detection threshold is crossed, a sleep apnea response is provided at step 354. The sleep apnea response may include a therapy delivery and/or reporting operations as described above. If sleep apnea is not detected according to a probability less than the detection threshold, sleep apnea monitoring may continue at step 352 according to the scheduled, triggered or continuous basis for which it is enabled.

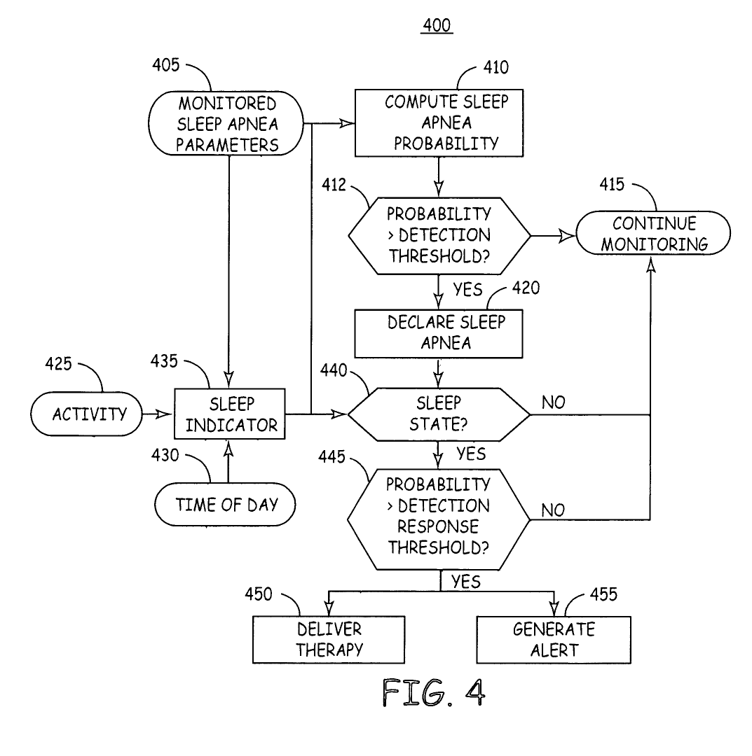

FIG. 4 is a flow chart summarizing steps included in a method for responding to a sleep apnea detection made according to the method 300 of FIG. 3. As described above, monitored sleep apnea parameters 405 are provided as input for computing a sleep apnea probability at step 410. A sleep state indicator 435 is determined using an activity sensor signal 425, the time of day 430, and/or one or more of the monitored sleep apnea parameters 405. Heart rate and minute ventilation are known to be low during sleep. The Q-T interval is known to be long during sleep. As such, any of these parameters may be used in detecting a sleep state. Other physiological signals may be used in detecting a sleep state, such as a posture signal. The sleep state indicator may be provided as input for computing the sleep apnea probability at step 410.

The sleep apnea probability is compared to a sleep apnea detection threshold at decision step 412. If the sleep apnea probability is greater than a detection threshold, sleep apnea is declared at step 420. If the sleep apnea probability is not greater than the detection threshold, sleep apnea monitoring continues at step 415.

After declaring a sleep apnea detection at step 420, one or more response conditions may be required prior to generating a sleep apnea response. In one embodiment, the condition of verifying a sleep state at decision step 440 may be required before generating a sleep apnea response. The sleep state may be verified according to sleep indicator 435. If the sleep state is not verified, sleep apnea monitoring continues at step 415 without delivering a sleep apnea response.

Another condition that may be required for delivering a sleep apnea response is a sleep apnea probability that exceeds a predetermined response threshold. The response threshold may be defined as a required magnitude of the sleep apnea probability. The response threshold may additionally include a minimal time duration over which the sleep apnea probability must continuously exceed the required magnitude. A unique response threshold may be set for different types of reporting or therapy delivery responses. A response threshold magnitude may be equal to or greater than the sleep apnea detection threshold. The response threshold may be relatively low for triggering storage of sleep apnea episode data and relatively higher for generating an alert or delivering a therapy.

If the sleep apnea probability exceeds a response threshold, the corresponding response is provided. In the example of FIG. 4, if the probability exceeds a response threshold for therapy delivery, the therapy is delivered at step 450. If the probability exceeds a response threshold for generating an alert, the alert is generated at step 455. If the response threshold requirement is not met for any of the enabled responses, sleep apnea monitoring continues at step 415.

A clinician may program the desired responses to be enabled or disabled in response to a sleep apnea detection and may program corresponding response thresholds for each of the enabled responses. Various responses that can be enabled by a clinician may include, but are not limited to, a patient alert transmitted from an IMD to an external home monitor or patient activator, a patient alert provided as a perceptible muscle stimulation or vibration, a patient alert provided as an audible sound (for example, to arouse the patient), a clinician alert provided via a communication network, e.g. through remote patient management system, or a sleep apnea therapy such as atrial overdrive pacing, or other neuromuscular stimulation.

Thus a medical device system and method have been described for detecting respiratory disturbances such as sleep apnea. It is recognized that one having skill in the art and the benefit of the teachings provided herein may conceive of numerous variations to the embodiments presented herein. The systems and methods described are intended to be illustrative embodiments of the invention and should not be construed as limiting with regard to the following claims.

What is claimed is:

1. A method, comprising:
sensing a plurality of physiological signals;
deriving a plurality of physiological parameters from the sensed signals;
computing a probability of a respiratory disturbance from the physiological parameters; and
detecting a respiratory disturbance when the computed probability exceeds a predefined detection threshold.

2. The method of claim 1 wherein the physiological signals comprise a cardiac electrical signal, a respiration signal, and a blood oxygen saturation signal.

3. The method of claim 1 wherein the physiological parameters comprise a heart rate variability, a Q-T interval variability, a respiration rate, a respiration depth, a minute ventilation, and a blood oxygen saturation.

4. The method of claim 1 wherein computing a probability of a respiratory disturbance comprises computing a weighted sum of a heart rate variability, a Q-T interval variability, a respiration rate, a respiration depth, a minute ventilation, and a blood oxygen saturation.

5. The method of claim 1 wherein computing a probability of a respiratory disturbance comprises determining a logical value for one or more of the physiological parameters by comparing the derived physiological parameter value to a predetermined threshold value.

6. The method of claim 1 wherein the respiratory disturbance is sleep apnea.

7. The method of claim 1 further comprising providing a response to the detected respiratory disturbance.

8. The method of claim 7 wherein the response comprises delivering a therapy.

9. The method of claim 8 wherein delivering a therapy comprises delivering atrial overdrive pacing.

10. The method of claim 7 wherein the response comprises reporting the detected respiratory disturbance.

11. The method of claim 7 further comprising:
determining a sleep state indicator; and
providing the response to the detected respiratory disturbance when the sleep state indicator is positive for detecting a sleeping state.

12. The method of claim 1 further comprising determining a sleep state for use in computing the probability of a respiratory disturbance.

13. A system, comprising:
a physiological sensor;
a processor for deriving a physiological parameter from a signal received from the physiological sensor and for computing a probability of a respiratory disturbance from the physiological parameter; and
a response module for controlling a response to a respiratory disturbance detection signal generated by the processor when the computed probability exceeds a detection threshold.

14. The system of claim 11 further comprising a therapy delivery module controlled by the response module.

15. The system of claim 11 further comprising an alert module controlled by the response module.

16. The system of claim 11 further comprising a communications module controlled by the response module.

17. A system, comprising:
means for sensing a plurality of physiological signals;
means for deriving a plurality of physiological parameters from the signals;
means for computing a probability of a respiratory disturbance from the plurality of physiological parameters;
means for detecting a respiratory disturbance episode using the computed probability; and
means for responding to the detected respiratory episode.

18. A computer readable medium for storing a set of instructions which when implemented in a system cause the system to:
sense a plurality of physiological signals;
derived a plurality of physiological parameters from the sensed signals; compute a probability of a respiratory disturbance using the physiological parameters; and
detect the respiratory disturbance when the computed probability crosses a predetermined detection threshold.
